(19) United States
(12) Patent Application Publication
(10) Pub. No.: US 2024/0099728 A1
(43) Pub. Date: Mar. 28, 2024

(54) METHOD AND APPARATUS FOR SURGICAL DRILL STOP

(71) Applicant: PHASOR HEALTH, LLC, Houston, TX (US)

(72) Inventors: Ray KING, Houston, TX (US); Ashok GOWDA, Bellaire, TX (US); Charles HOUSSIERE, Houston, TX (US)

(21) Appl. No.: 18/264,405

(22) PCT Filed: Feb. 5, 2022
(86) PCT No.: PCT/US2022/015387

§ 371 Date: Aug. 4, 2023

(51) Int. Cl. A61B 17/16; A61B 90/00

(52) U.S. Cl. CPC A61B 17/1633; A61B 90/03; A61B 2090/035

(57) ABSTRACT

The present invention relates generally to methods and systems for an adjustable drill stop, which has additional optional features of tissue retraction, visualization via transparent or semi-transparent material and/or window(s) on its size, precise gradation of depth by rotation indicated on side of the device, windows on the side for irrigation while drilling, and ability to utilize this device in setting of surgical drilling, in a drill which cannot be easily “overpowered” by typically applied forces (e.g. downward pressure by typical user).

### CROSS-REFERENCE TO RELATED APPLICATIONS

This Patent Cooperation Treaty international application claims the benefit and priority of U.S. provisional patent application Ser. No. 63/146,063, filed Feb. 5, 2021, entitled “METHOD AND APPARATUS FOR SURGICAL DRILL STOP”, the contents of which is hereby incorporated by reference in its entirety.

### FIELD OF THE INVENTION

This invention relates generally to a drill stop which prevents a surgical drill from penetrating too far or plunging beyond a desirable depth, such as into a tissue (e.g. into the bone or beyond the bone), an organ (e.g. the brain) or a blood vessel/nerve/organ/other tissue, and further relates to methods and systems for using such a drill stop.

### BACKGROUND OF THE INVENTION

Surgical drills powered by batteries, pneumatically, or electrically function to penetrate tissue, but once the drill has reached or traversed the area desired to drill, they typically employ a stopper to prevent excessive travel beyond the intended length or depth. To facilitate this, current manual or automatic drills (e.g. for neurosurgical or orthopedic procedures) have a drill stop. The most common version, for neurosurgical procedures, are found in the “Cranial Access Kit” for External Ventricular Drain (EVD). The manual drill is typically accompanied by adjustable drill stops which comprise a plastic ring with a hole drilled on its side, through which a set screw is placed (and tightens or loosens manually, such as via an Allen key). This drill stop has a goal of preventing undesirable plunging past the intended target, so can be so adjusted by the user with the goal to accomplish this.

In a percentage of cases (ranging from 0.5% to 10%, however, drills “plunge” beyond the intended depth, and hence a drill stop that cannot be overpowered (e.g. the United States Food and Drug Administration (FDA) standard of predicate devices is five pounds of weight able to be withstood) easily is desirable, to further minimize or completely negate plunging risk.

### SUMMARY OF THE INVENTION

This invention relates generally to a drill stop which prevents a surgical drill from penetrating too far or plunging beyond a desirable depth, such as into a tissue (e.g. into the bone or beyond the bone), an organ (e.g. the brain) or a blood vessel/nerve/organ/other tissue, and further relates to methods and systems for using such a drill stop. In general, a drill stop may include a cylindrical member through which a drill bit from a surgical drill may pass through. The cylindrical member may generally be wider in diameter than the drill bit to a degree that the cylindrical member may abut against the surface being drilled to stop penetration of the drill bit during use after a given distance of travel. The drill stop may generally be utilized by attachment to the drill body, attachment to an existing primary drill stop, or held against manually or attached to a body surface during drilling. The drill stop may be constructed of any appropriate material, such as plastic, metal, composite, and/or a combination thereof.

In one exemplary aspect, the drill stop may include an adjustability feature to change the amount of depth before the stop abuts a surface to control the depth of penetration of the drill bit. In some embodiments, the adjustability feature includes a depth adjusting insert that may interface with the cylindrical member of the drill stop and act to adjust the overall length of the drill stop to affect the amount of depth of penetration when using a drill bit. The adjusting insert may, for example, thread into corresponding threads in the cylindrical member and the overall length of the drill stop may then be adjusted by rotating the adjusting insert in the appropriate direction to thread or unthread it from the cylindrical member. In general, the threading may produce enough friction and/or other resistance such that the adjusting insert remains at the given degree of insertion into the cylindrical member during use. The drill stop may thus remain adjustable, but may be tightened to its desired position without relying upon other forms of locking, such as a side-bracing set screw. This may be desirable such that greater than five pounds of pressure (e.g. the metric used by companies for FDA clearance) is not adequate to displace it—ideally, a drill stop capable of withstanding about five to ten times this weight or more, making it impractical for a drill to plunge may be desirable. In some embodiments, an additional locking mechanism, such as a locking collar, a set screw or any other appropriate locking mechanism may also be included for added assurance in maintaining the desired depth setting.

In some embodiments, the threading may be designed such that the depth setting is metered based on the number of full rotations of the adjusting insert. For example, a full rotation may increase or lower, depending on the direction, the overall length of the drill stop by a set amount, such as 1 mm or any other desirable amount of depth.

In some embodiments, the drill stop may include markings or other forms of graduations such that a user may visually assess the depth setting.

In another aspect of the invention, the drill stop may include a visualization and/or access feature(s). In general, a visualization feature may provide the user with a view of the interior of the drill stop, such as, for example, to see the immediate area being drilled. In some embodiments, a window or other aperture may be utilized as a visualization feature. A window or aperture may also provide access to the area, such as for irrigation during drilling. The window or aperture may also be generally placed proximal to the end of the drill stop that abuts the surface during drilling. In some embodiments, the window or aperture may be positioned on the drill stop such that it is elevated off the surface during use, for example, to prevent tissue from blocking or entering the window or aperture, such as the scalp or skin.

In some embodiments, at least a portion of the drill stop may be constructed from a transparent or translucent material such that a user may visualize through the material of the drill stop.

In another aspect of the invention, the drill stop may include features for providing tissue retraction during use. For example, it may be desirable to prevent tissue from entering the drilling area, such as to prevent tissue from contacting the drill bit and being damaged/chafed. In some embodiments, the drill stop may include a lip or other protruding formation that may, for example, be utilized to push away tissue and/or be inserted under the tissue to retract it away from the drilling area, such as, for example, inserting under skin/scalp.

In another aspect of the invention, the drill stop may include features for aiding in the user holding and/or stabilizing the drill stop during use. In some embodiments, the drill stop may include finger impressions or other gripping features such that the user may hold it as needed, for stabilization; alternatively, it may be screwed onto the primary drill stop or the drill housing, or used free-hand without stabilization (e.g. with manual or power drills).

The present invention together with the above and other advantages may best be understood from the following detailed description of the embodiments of the invention and as illustrated in the drawings. The following description, while indicating various embodiments of the invention and numerous specific details thereof, is given by way of illustration and not of limitation. Many substitutions, modifications, additions or rearrangements may be made within the scope of the invention, and the invention includes all such substitutions, modifications, additions or rearrangements.

### BRIEF DESCRIPTION OF THE DRAWINGS

FIG. 1 illustrates a drill stop of the present invention;

FIG. 1a illustrates the cylindrical member of the drill stop;

FIG. 2 illustrates a drill stop interfaced with a surgical drill; and

FIG. 3 illustrates a prior art surgical drill with a primary drill stop.

### DETAILED DESCRIPTION OF THE INVENTION

The detailed description set forth below is intended as a description of the presently exemplified systems, devices and methods provided in accordance with aspects of the present invention and are not intended to represent the only forms in which the present invention may be prepared or utilized. It is to be understood, rather, that the same or equivalent functions and components may be accomplished by different embodiments that are also intended to be encompassed within the spirit and scope of the invention. Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplary methods, devices and materials are now described. All publications mentioned herein are incorporated herein by reference for the purpose of describing and disclosing, for example, the designs and methodologies that are described in the publications which might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

This invention relates generally to a drill stop which prevents a surgical drill from penetrating too far or plunging beyond a desirable depth, such as into a tissue (e.g. into the bone or beyond the bone), an organ (e.g. the brain) or a blood vessel/nerve/organ/other tissue, and further relates to methods and systems for using such a drill stop.

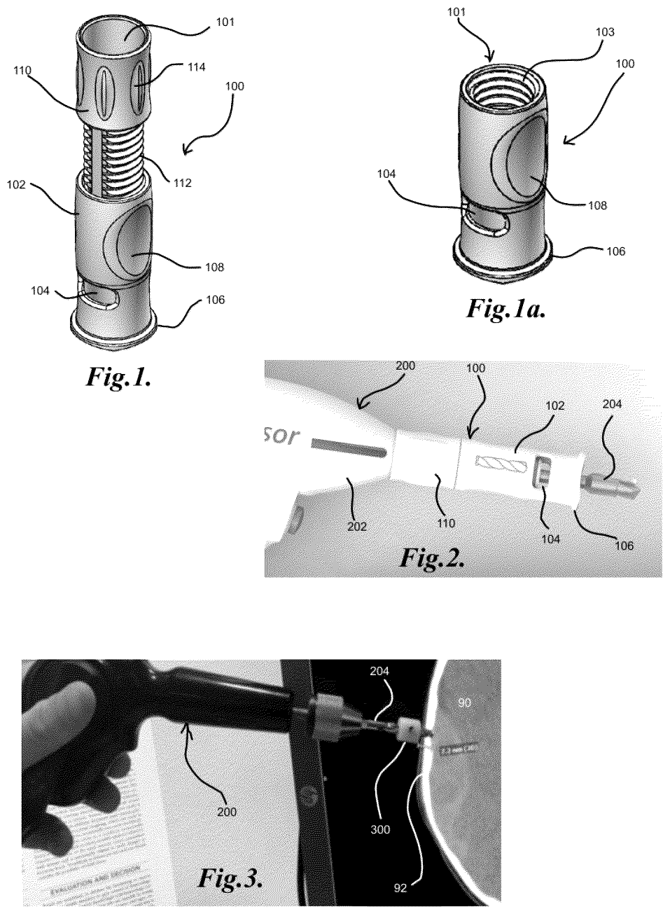

In general, as illustrated in FIG. 1, a drill stop 100 may include a cylindrical member 102, shown more specifically in FIG. 1a, through which a drill bit from a surgical drill may pass through via a channel 101. The cylindrical member 102 may generally be wider in diameter than the drill bit to a degree that the cylindrical member 100 may abut against the surface being drilled to stop penetration of the drill bit during use after a given distance of travel. FIG. 2 illustrates a surgical drill 200 with a drill body 202 and drill bit 204 shown as significantly narrower than the cylindrical member 102. The drill stop 100 may generally be utilized by attachment to the drill body 202, attachment to an existing primary drill stop, such as the primary drill stop 300 illustrated in FIG. 3 against a body part 90 (e.g. illustrated as against a skull 92), or held against manually or attached to a body surface during drilling.

The drill stop 100 may be constructed of any appropriate material, such as plastic, metal, composite, and/or a combination thereof. For example, in general, the various components of the drill stop 100 may be made from appropriate materials, such as polymers, metals, composites, and/or any other appropriate material or combinations thereof. Suitable polymers may include, but are not limited to, polyethylene; polypropylene; polybutylene; polystyrene; polyester; polytetrafluoroethylene (PTFE); acrylic polymers; polyvinylchloride; Acetal polymers such as polyoxymethylene or Delrin (available from DuPont Company); natural or synthetic rubber; polyamide, or other high temperature polymers such as polyetherimide like ULTEM®, a polymeric alloy such as Xenoy® resin, which is a composite of polycarbonate and polybutyleneterephthalate, Lexan® plastic, which is a copolymer of polycarbonate and isophthalate terephthalate resorcinol resin (all available from GE Plastics); liquid crystal polymers, such as an aromatic polyester or an aromatic polyester amide containing, as a constituent, at least one compound selected from the group consisting of an aromatic hydroxycarboxylic acid (such as hydroxybenzoate (rigid monomer), hydroxynaphthoate (flexible monomer), an aromatic hydroxyamine and an aromatic diamine, (exemplified in U.S. Pat. Nos. 6,242,063, 6,274,242, 6,643,552 and 6,797,198, the contents of which are incorporated herein by reference), polyesterimide anhydrides with terminal anhydride group or lateral anhydrides (exemplified in U.S. Pat. No. 6,730,377, the content of which is incorporated herein by reference) or combinations thereof. Some of these materials are recyclable or may be made to be recyclable. Compostable or biodegradable materials may also be used and may include any biodegradable or biocompostable polyesters such as a polylactic acid resin (comprising L-lactic acid and D-lactic acid) and polyglycolic acid (PGA), polyhydroxyvalerate/hydroxybutyrate resin (PHBV) (copolymer of 3-hydroxy butyric acid and 3-hydroxy pentanoic acid (3-hydroxy valeric acid) and polyhydroxyalkanoate (PHA) copolymers, and polyester/urethane resin. Some non-compostable or non-biodegradable materials may also be made compostable or biodegradable by the addition of certain additives, for example, any oxo-biodegradable additive such as D2W™ supplied by (Symphony Environmental, Borehamwood, United Kingdom) and TDPA® manufactured by EPI Environmental Products Inc. Vancouver, British Columbia, Canada. Compostable or biodegradable materials may also be desirable for disposable versions or portions of the drill stop 100. In addition, any polymeric composite such as engineering prepregs or composites, which are polymers filled with pigments, carbon particles, silica, glass fibers, or mixtures thereof may also be used. For example, a blend of polycarbonate and ABS (Acrylonitrile Butadiene Styrene) may be used for the housing. For further example, carbon-fiber and/or glass-fiber reinforced plastic may also be used. Useful metals or metallic materials may include metal and metal alloys such as aluminum, steel, stainless steel, nickel titanium alloys, shape memory alloys and so on. In general, for certain applications which involve imaging, the drill stop 100 may be made from magnetic resonance imaging (MRI) compatible or X-ray/computerized tomography (CT) compatible materials, such as polymers, titanium, aluminum, etc.

In one exemplary aspect, the drill stop 100 may include an adjustability feature to change the amount of depth before the stop abuts a surface to control the depth of penetration of the drill bit 204. In some embodiments, the adjustability feature includes a depth adjusting insert that may interface with the cylindrical member of the drill stop and act to adjust the overall length of the drill stop to affect the amount of depth of penetration when using a drill bit, as illustrated with the adjusting insert 110 in FIGS. 1 and 2. The adjusting insert 110 may, for example, thread into corresponding threads in the cylindrical member 102 and the overall length of the drill stop 100 may then be adjusted by rotating the adjusting insert 110 in the appropriate direction to thread or unthread it from the cylindrical member 102. FIGS. 1 and 2 illustrate a threading adjusting insert 110 that threads into cylindrical member 102 via threads 112 and 103, respectively. In general, the threading, such as between the threads 103, 112, may produce enough friction and/or other resistance such that the adjusting insert 110 remains at the given degree of insertion into the cylindrical member 102 during use. The drill stop 100 may thus remain adjustable, but may be tightened to its desired position without relying upon other forms of locking, such as a side-bracing set screw. This may be desirable such that greater than five pounds of pressure (e.g. the FDA standard) is not adequate to displace it—ideally, a drill stop 100 capable of withstanding five to ten times this weight or more, making it impractical for a drill to plunge may be desirable. In some embodiments, an additional locking mechanism, such as a locking collar, a set screw or any other appropriate locking mechanism may also be included for added assurance in maintaining the desired depth setting.

In some embodiments, the threading, such as between threads 103, 112, may be designed such that the depth setting is metered based on the number of full rotations of the adjusting insert 110. For example, a full rotation may increase or lower, depending on the direction, the overall length of the drill stop 100 by a set amount, such as 1 mm or any other desirable amount of depth.

In some embodiments, the drill stop 100 may include markings or other forms of graduations such that a user may visually assess the depth setting.

In another aspect of the invention, the drill stop 100 may include a visualization and/or access feature(s). In general, a visualization feature may provide the user with a view of the interior of the drill stop, such as, for example, to see the immediate area being drilled. In some embodiments, a window or other aperture may be utilized as a visualization feature. A window or aperture may also provide access to the area, such as for irrigation during drilling. The window or aperture may also be generally placed proximal to the end of the drill stop that abuts the surface during drilling. In some embodiments, the window or aperture may be positioned on the drill stop such that it is elevated off the surface during use, for example, to prevent tissue from blocking or entering the window or aperture, such as the scalp or skin. FIGS. 1, 1a and 2 illustrate a window 104 for visualization/access.

In some embodiments, at least a portion of the drill stop 100 may be constructed from a transparent or translucent material such that a user may visualize through the material of the drill stop 100.

In another aspect of the invention, the drill stop 100 may include features for providing tissue retraction during use. For example, it may be desirable to prevent tissue from entering the drilling area, such as to prevent tissue from contacting the drill bit and being damaged/chafed. In some embodiments, the drill stop 100 may include a lip or other protruding formation, such as the lip 106 illustrated in FIGS. 1, la and 2, that may, for example, be utilized to push away tissue and/or be inserted under the tissue to retract it away from the drilling area, such as, for example, inserting under skin/scalp.

In another aspect of the invention, the drill stop 100 may include features for aiding in the user holding and/or stabilizing the drill stop 100 during use. In some embodiments, the drill stop 100 may include finger impressions or other gripping features, as illustrated with gripping features 108 and 114 in FIGS. 1 and 1a, such that the user may hold it as needed, for stabilization; alternatively, it may be screwed onto the primary drill stop 300 or the drill housing 202, or used free-hand without stabilization (e.g. with manual or power drills).

### EXAMPLES: USE OF DRILL STOP

External Ventricular Drain (EVD, or ventriculostomy drain) placement involves drilling a hole in the skull, taking care to avoid not drilling too deep (as the dura or brain may be penetrated, resulting in permanent injury)—with present drilling being somewhat controlled by a “depth stop” which is a plastic or nylon cannulated cylinder and a metal set screw on its side tightened to abut the metal drill bit, this is highly user dependent (in terms of how tightly or loosely the set screw was originally tightened, and how much downward pressure was placed on the drill when drilling)—with 5 pounds being the threshold of predicate devices (such as manual drills) required for FDA clearance. Unfortunately, plunging occurs (reflected by personal experiences and the published peer-reviewed literature) for this scenario and/or in power drills (even those purported to be typically “self-stopping”). In orthopedic cases including extremity or spine cases, similar protection against drilling too far may be required, to avoid injury to distal blood vessels, nerves, or other tissue. Creating the entire structure with gradations to measure the depth, as well as of a clear material vs. opaque, would allow visualization inside; in addition, a longer version of this could be used for spine procedures whereby the pedicle screw may be placed but length of drill limited to not exit the vertebral body anteriorly (similarly for kyphoplasty).

Although the invention has been described with respect to specific embodiments thereof, these embodiments are merely illustrative, and not restrictive of the invention. The description herein of illustrated embodiments of the invention, including the description in the Abstract and Summary, is not intended to be exhaustive or to limit the invention to the precise forms disclosed herein (and in particular, the inclusion of any particular embodiment, feature or function within the Abstract or Summary is not intended to limit the scope of the invention to such embodiment, or feature, while functionally limited to drilling). Rather, the description is intended to describe illustrative embodiments, features and functions in order to provide a person of ordinary skill in the art context to understand the invention without limiting the invention to any particularly described embodiment, feature or function, including any such embodiment feature or function described in the Abstract or Summary. While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention, while maintaining its use to the indication of drilling.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, process, article, or apparatus.

1. A drill stop for a surgical drill comprising:
a cylindrical member having a channel adapted to receive a drill bit of a surgical drill therethrough; and an adjusting insert adapted to interface with said cylindrical member to alter the length of said drill stop; wherein said drill stop is adapted to withstand an applied force of at least five pounds of pressure.

2. The drill stop of claim 1, wherein said adjusting insert interfaces with said cylindrical member via threadings.

3. The drill stop of claim 1, further comprising a visualization aperture.

4. The drill stop of claim 1, further comprising a tissue retraction feature.

5. A method for using a drill stop with a surgical drill comprising:
providing a drill stop comprising:
a cylindrical member having a channel adapted to receive a drill bit of a surgical drill therethrough; and
an adjusting insert adapted to interface with said cylindrical member to alter the length of said drill stop;
adjusting said drill stop via said adjusting insert to set a desired drill depth;
inserting a drill bit of a surgical drill through said channel; and
actuating said drill bit to penetrate a body tissue of a patient at a penetration site until said surgical drill abuts against said drill stop and said drill stop abuts against a surface of said body tissue surrounding said penetration site to stop the penetration of said drill bit;
wherein said drill stop is adapted to withstand an applied force of at least five pounds of pressure.
